(19) United States
(12) Patent Application Publication
(10) Pub. No.: US 2020/0049002 A1
(43) Pub. Date: Feb. 13, 2020

(54) CORELESS INJECTIVITY TESTING METHOD

(71) Applicant: THE SASKATCHEWAN RESEARCH COUNCIL, Saskatoon (CA)

(72) Inventors: Petro NAKUTNYY, Regina (CA); Peng LUO, Regina (CA); Kelvin Dean KNORR, Regina (CA); Michael John CRABTREE, Saskatoon (CA)

(21) Appl. No.: 16/485,722

(22) PCT Filed: Feb. 16, 2018
(86) PCT No.: PCT/CA2018/050179

§ 371 Date: Aug. 13, 2019

Related U.S. Application Data
(60) Provisional application No. 62/460,271, filed on Feb. 17, 2017.

(51) Int. Cl. E21B 49/00; E21B 21/06; G01N 33/24; G01N 1/34

(52) U.S. Cl. CPC E21B 49/005; E21B 21/066; E21B 43/20; G01N 1/34; G01N 33/241

(57) ABSTRACT

A coreless injectivity method. Drill cuttings from drilling a well into a subterranean formation rock are pressed into a compressed core plug and the compressed core plug subjected to injectivity testing. The injectivity testing of the compressed core plug is representative of the injectivity of the subterranean formation, so the injectivity of the subterranean formation can be determined. The compressed core plug can also be used to conduct relative permeabilities and displacement studies similar to the actual reservoir core plugs.

### CROSS REFERENCE TO RELATED APPLICATIONS

This application claims the benefit of priority of U.S. Provisional Patent Application No. 62/460,271 filed Feb. 17, 2017, which is hereby incorporated by reference.

### FIELD

The present disclosure relates generally to characterization of a subterranean formation. More particularly, the present disclosure relates to determining the injectivity of a fluid into a subterranean formation comprising siliciclastic rock, including arenite, wackestone and/or mudstone.

### BACKGROUND

The production of oil in gas from a subterranean formation may include injection of a fluid, such as water, into a well. One such circumstance is a water flood.

One must know properties of the subterranean formation, in order to reduce the risk of incompatibilities between the injection fluid and the subterranean formation rock and fluids.

This can be achieved by obtaining a core sample during the drilling of the well or by obtaining a core sample by drilling a dedicated core from another portion of the subterranean formation.

Coreflooding, as described for example in the Schlumberger® Oilfield Glossary at http://www.glossary.oilfield.slb.com/Terms/c/coreflooding.aspx is a laboratory test in which a fluid or combination of fluids is injected into a sample of rock. Objectives include measurement of permeability, relative permeability, saturation change, formation damage caused by the fluid injection, or interactions between the fluid and the rock.

An injectivity test may be performed on the core sample to determine the injectivity of a fluid into the subterranean formation.

Permeability is a measure of the ability of a porous medium to allow fluids to pass through it. Porosity is a percentage of void space in a rock. Relative permeability of a rock to a certain fluid phase is a ratio of the effective permeability for that phase to the absolute permeability. All these are important characteristics of the formation and can be used to estimate the amount of oil that can be stored in the formation and the ability of the formation rock to transport the oil towards the production wells. Displacement efficiency can be used to estimate the ability of the given process to mobilize oil in the formation.

Obtaining a core sample requires a core drill, in order to obtain a cylindrical section of the subterranean formation. While valuable information may be obtained by analyzing the core sample, relatively few core samples are obtained due to the high cost. As a result, most wells have no core samples taken. For such wells, one must extrapolate available data or merely accept the risk of not having injectivity test results.

A method for handling, processing and disposing of drill cuttings is disclosed in US 200310006202A1 (Boutte et al.).

An apparatus for sampling drill hole cuttings is disclosed in U.S. Pat. No. 6,845,657 (Williams).

An apparatus and process for formation gas pore pressure evaluation on drilling cuttings samples is disclosed in US 2005/0066720A1 (Zamfes) and/or U.S. Pat. No. 7,418,854 (Zamfes) and/or US 2009/0038389A1 (Zamfes).

A drilling cutting analyzer system and methods of applications is disclosed in US 2005/0082468A1 (Zamfes).

A mini core in drilling samples for high resolution formation evaluation on drilling cuttings samples is disclosed in US 2005/0072251A1 (Zamfes) and/or US 2007/0175285A1 (Zamfes) and/or US 2009/0038853A1 (Zamfes).

A method and apparatus for on-site drilling cuttings analysis is disclosed in WO 2010/000055A1 (Zamfes/Canadian Logging Systems Corp.).

A method and apparatus for determining physical properties of solid materials suspended in a drilling fluid is disclosed in WO 2013/162400A1 (Vladimirovich/Siemens Aktiengesellschaft).

An apparatus and method for separating and weighing cuttings received from a wellbore while drilling is disclosed in U.S. Pat. No. 9,297,225 (Nesheim et al.).

A method and apparatus for analyzing drill cuttings is disclosed in U.S. Pat. No. 5,571,962 (Georgi et al.).

A method for finding and evaluating rock specimens having classes of fluid inclusions for oil and gas exploration is disclosed in U.S. Pat. No. 5,241,859 (Smith).

An apparatus for collecting and washing well cutting is disclosed in U.S. Pat. No. 3,563,255 (Morris).

An apparatus and methodology for measuring properties of microporous material at multiple scales is disclosed in WO 2014/123973A1 (Chertov et al./Schlumberger Canada Ltd.) and/or WO 2014/123966 (Chertov et al./Schlumberger Canada Ltd.) and/or EP 2954307A1 (Chertov et al./Schlumberger Canada Ltd.).

A method and device for evaluating physical parameters of an underground reservoir from rock cuttings taken therefrom is disclosed in U.S. Pat. No. 7,082,812 (Lenormand et al.).

However, none of the above background art address the problem of assessing a bulk petrophysical property of a siliciclastic subterranean formation rock wherein few (or zero) formation core plugs are available. The present disclosure solves the problem by providing a compressed core plug that may be evaluated to provide one or more petrophysical property of the siliciclastic subterranean formation, including but not limited to core flooding, for example injectivity testing.

It is, therefore, desirable to provide a coreless injectivity testing method.

### SUMMARY

It is an object of the present disclosure to obviate or mitigate at least one disadvantage of previous injectivity testing methods.

The present disclosure provides a coreless injectivity method. Drill cuttings from drilling a well into a subterranean formation rock are pressed into a compressed core plug and the compressed core plug subjected to injectivity testing. The injectivity testing of the compressed core plug is representative of the injectivity of the subterranean formation, so the injectivity of the subterranean formation can be determined. The compressed core plug can also be used to conduct relative permeabilities and displacement studies similar to the actual reservoir core plugs.

In a first aspect, the present disclosure provides a method for assessing a siliciclastic subterranean formation rock petrophysical property, including providing drill cuttings from the siliciclastic subterranean formation, cleaning the drill cuttings, forming a compressed core plug from the drill cuttings, installing the compressed core plug into a coreholder, and injecting a test fluid into the compressed core plug in order to assess a compressed core plug petrophysical property, wherein the compressed core plug petrophysical property represents the corresponding siliciclastic subterranean formation rock petrophysical property, prior to injecting the test fluid into the compressed core plug.

In an embodiment disclosed, the siliciclastic subterranean formation rock comprises arenite, wackestone, mudstone, or combinations thereof.

In an embodiment disclosed, the petrophysical property is selected from the group consisting of fluid injectivity; in-situ displacement performance; displacement studies; displacement efficiency; relative permeabilities; multi-phase relative permeabilities; permeability; porosity; and combinations thereof.

In an embodiment disclosed, the cleaning includes washing the drill cuttings with a KCl solution, wherein the drill cuttings were obtained from a drilling operation using a water-based drilling mud.

In an embodiment disclosed, the cleaning includes washing the drill cuttings with a solvent, wherein the drill cuttings were obtained from a drilling operation using an oil-based drilling fluid.

In an embodiment disclosed, the solvent comprises benzene, toluene, ethylbenzene, or xylene.

In an embodiment disclosed, the solvent is toluene.

In an embodiment disclosed, the method further includes removing at least a portion of bentonite from the drill cuttings, if any, before cleaning.

In an embodiment disclosed, the removing comprises gravity separation.

In an embodiment disclosed, the method further includes conditioning the drill cuttings prior to forming the compressed core plug.

In an embodiment disclosed, the conditioning includes drying the drill cuttings.

In an embodiment disclosed, the conditioning further comprises packing the drill cuttings with a packing fluid.

In an embodiment disclosed, the packing fluid comprises a solvent.

In an embodiment disclosed, the solvent is an organic or aromatic solvent.

In an embodiment disclosed, the organic or aromatic solvent comprises benzene, toluene, ethylbenzene, or xylene.

In an embodiment disclosed, the organic or aromatic solvent is toluene.

In an embodiment disclosed, forming the compressed core plug comprises placing a quantity of the drill cuttings into a form and applying a pressure to the drill cuttings to compress the drill cuttings to form the compressed core plug.

In an embodiment disclosed, the pressure is a selected pressure.

In an embodiment disclosed, the selected pressure is in the range of about 3,000 psi to about 10,000 psi.

In an embodiment disclosed, the selected pressure is applied for a selected time.

In an embodiment disclosed, the selected pressure is substantially 5,000 psi and the selected time is substantially 30 minutes.

In an embodiment disclosed, the method further includes drying the compressed core plug before injecting the test fluid.

In an embodiment disclosed, the petrophysical property includes injectivity testing, and provides a subjective injectivity analysis, selected from the group consisting of observed swelling; maximum injection pressure; substantially zero fluid flow rate; compatibility of the test fluid and the compressed core plug; and combinations thereof.

In an embodiment disclosed, the petrophysical property includes injectivity testing, and provides a quantitative injectivity analysis, selected from the group consisting of fluid flow rate; change in fluid injection pressure; change in fluid production pressure; change in differential pressure along the compressed core plug; confining pressure; and combinations thereof.

In an embodiment disclosed, the method further includes determining a corresponding formation petrophysical property of the siliciclastic subterranean formation from the compressed core plug petrophysical property.

In an embodiment disclosed, the method further includes reducing a formation core plug obtained from the siliciclastic subterranean formation to obtain the drill cuttings.

In an embodiment disclosed, the method further includes generating a numerical model, for use with injectivity test results from a compressed core plug, to conform or match the results.

In an embodiment disclosed, the siliciclastic subterranean formation rock has a clay content of about 5 wt. percent or more.

In an embodiment disclosed, the clay content is about 10 wt. percent or more.

In an embodiment disclosed, the clay content is about 15 wt. percent or more.

In an embodiment disclosed, installing the compressed core plug into the coreholder includes stacking a plurality of compressed core plugs into the coreholder.

In a further aspect, the present disclosure provides a method for assessing a petrophysical property of a siliciclastic subterranean formation rock having a clay content of about 5 wt. percent or more, including providing drill cuttings from the siliciclastic subterranean formation, cleaning the drill cuttings, with KCl if the drill cuttings include water-based drilling fluid, or with a solvent if the drill cuttings include oil-based drilling fluid, drying the drill cuttings, packing the drill cuttings with a packing fluid, preferably toluene, forming a compressed core plug from the drill cuttings by placing a quantity of the drill cuttings into a form and applying a pressure to compress the drill cuttings to form the compressed core plug, drying the compressed core plug, installing the compressed core plug into a coreholder, and injecting a test fluid into the compressed core plug while measuring an injected volume and a differential pressure, wherein the injectivity of the test fluid into the compressed core plug is representative of the injectivity of the test fluid into the siliciclastic subterranean formation rock.

In an embodiment disclosed, installing the compressed core plug into the coreholder includes stacking a plurality of compressed core plugs into the coreholder.

In a further aspect, the present disclosure provides a method of forming a compressed core plug for assessing a siliciclastic subterranean formation rock petrophysical property, including providing drill cuttings from the siliciclastic subterranean formation, cleaning the drill cuttings with a KCl solution if the drill cuttings include water-based drilling fluid or with a solvent if the drill cuttings include oil-based drilling fluid, drying the drill cuttings, packing the drill cuttings with a packing fluid, preferably toluene, forming a compressed core plug from the drill cuttings by placing a quantity of the drill cuttings into a form and applying a pressure to compress the drill cuttings to form the compressed core plug, drying the compressed core plug, and wherein the compressed core plug is adapted to undergo core flooding evaluation.

In an embodiment disclosed, the method further includes installing the compressed core plug into a coreholder.

In an embodiment disclosed, installing the compressed core plug into the coreholder includes stacking a plurality of compressed core plugs into the coreholder.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is an example of a cleaned drill cuttings sample.

FIG. 2 is an example of a compressed core plug of the present disclosure, prepared from drill cuttings.

FIG. 3 illustrates exemplary injectivity test results for a compressed core plug of the present disclosure, using a variety of injection brines with different KCl concentrations.

FIG. 4 illustrates exemplary injectivity test results for a compressed core plug of the present disclosure, using a variety of injection brines with different proportions from drill cuttings.

FIG. 5 illustrates exemplary injectivity test results for a reservoir core plug, using a variety of injection brines with different proportions.

### DETAILED DESCRIPTION

Generally, the present disclosure provides a method for coreless injectivity testing.

A typical earth rock drilling operation involves providing a drill bit on a drill string and rotating the drill bit to form a wellbore into the subterranean formation. Drilling fluid (also known as drilling mud) is pumped from the surface, down the interior of the drill string, across the face of the drill bit to cool the drill bit and to remove drill cuttings, and returns up to the surface with the drill cuttings via the annulus between the exterior of the drill string and the wellbore. At the surface, the drill cuttings and the drilling fluid are separated through a vibrating sieve (commonly referred to as a shale shaker) or by other means. The drilling fluid is circulated back down the interior of the drill string to repeat the process, and the drill cuttings are disposed of.

In the presently disclosed method, drill cuttings are collected during the drilling of the wellbore and as described below used to determine one or more properties of the subterranean formation. The drill cuttings may, for example, be collected from the shale shaker, but may be otherwise collected from the drilling operation. During the collection, the drilling depth is recorded so that the source depth of the drill cuttings is known. In an embodiment disclosed, drill cuttings may be collected periodically, for example every meter of depth when the subterranean formation is of interest. In an embodiment disclosed, about 4-5 kg of drill cuttings are collected per meter of formation drilled.

The collected drill cuttings are subjected to conditioning. The conditioning may include cleaning, drying, packing or combinations thereof.

The cleaning may include gravity separation of bentonite in drilling mud from drill cuttings.

If the drill cuttings are from a well drilled with a water-based drilling mud, the cleaning may include addition or washing with water containing KCl (potassium chloride), such as about 3 wt % KCl. If the drill cuttings are from a well drilled with an oil-based (invert) drilling mud, the cleaning may include addition or washing with a solvent, such as an organic solvent, such as benzene, toluene, ethylbenzene, or xylene. The solvent is preferably toluene.

The cleaned drill cuttings are then dried. The drying may include placing the drill cuttings into a constant humidity oven at a selected temperature. The constant humidity oven may be at about 60 degrees Celsius and about 45 percent humidity.

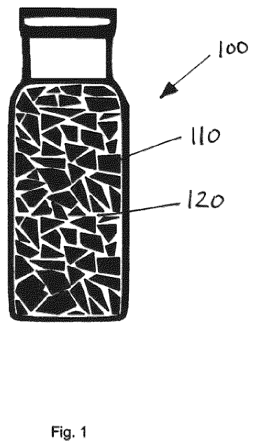

FIG. 1 is an example of drill cuttings 100 that have been cleaned and dried. The drill cuttings (100) may, for example, include formation rock (110) and clay (120).

The drill cuttings (100) are packed, compressed to form a compressed core plug (130), and dried.

The packing may include wetting or saturating the drill cuttings (100) with a packing fluid. The packing fluid may be a solvent, such as an aromatic or organic solvent, such as benzene, toluene, ethylbenzene, or xylene. The packing fluid is preferably toluene. The packing fluid, for example toluene, helps to keep the cleaned drill cuttings together during compression.

To form the compressed core plug (130), the packed drill cuttings may be placed into a form, such as a cylindrical pipe of a selected diameter, and a press, for example a hydraulic press, used to apply a pressure to the drill cuttings to form the compressed core plug (130). In an embodiment disclosed, a pressure of about 5000 psi is applied. In an embodiment disclosed, the pressure is applied for a selected amount of time. In an embodiment disclosed, the selected amount of time is about 30 minutes.

In an embodiment disclosed, the compressed core plug (130) has an air permeability and air porosity that is comparable with that of actual reservoir core plug. The pressure or time or both may vary for different formations/reservoirs.

The drying may include placing the compressed core plug (130) into a constant humidity oven at a selected temperature. The constant humidity oven may be at about 60 degrees Celsius and about 45 percent humidity.

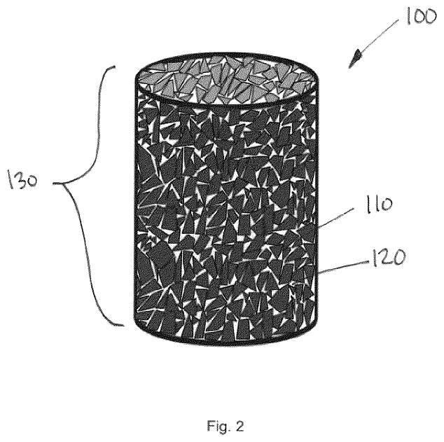

FIG. 2 is an example of a compressed core plug (130).

In an embodiment disclosed, the compressed core plug (130) may be sized to match a conventional core sample (e.g. real/actual reservoir core plug), so that the compressed core plug (130) may be used with readily available equipment for storing, handling, and testing of core samples. In an embodiment disclosed, the compressed core plug (130) is configured to be installed into a coreholder. Suitable coreholder equipment is known to one skilled in the art. In an embodiment disclosed, one or more compressed core plugs (130) may be stacked into a coreholder. In an embodiment disclosed, the compressed core plug may be cut or otherwise reduced in size to fit a coreholder. In an embodiment disclosed, the compressed core plug (130) is substantially cylindrical, substantially 1.5 inches in diameter and about 1.5 to 2 inches long. Any equipment suitable for coreflooding of an actual core (e.g. real/actual reservoir core plug) may be used for coreflooding the compressed core plug (130). One example of a suitable commercially available coreflooding system is the Core Flooding System 700 Bar (CFS 700) by Core Laboratories®/Sanchez Technologies®. Information is available at: http://www.corelab.com/sanchez/enhanced-oil-recovery/cfs-700. Other suitable coreflooding equipment is known to one skilled in the art.

The compressed core plug (130) may be subjected to coreflooding and other testing, measurement, and/or analysis to provide results that are comparable to that of an actual reservoir plug (e.g. real/actual reservoir core plug) and/or the subterranean formation from which the drill cuttings originated.

Just as one would conduct injectivity testing on a reservoir core plug, injectivity testing may be conducted on the compressed core plug (130).

In an injectivity test, a selected test fluid is injected into the compressed core plug (130) while the injection pressure and injection rate are recorded, as well as subjective observations made. The injectivity testing of the compressed core plug (130) may be used in order to determine the corresponding injectivity of the test fluid into the subterranean formation from which the drill cuttings were obtained.

A potassium chloride (KCl) solution may be used for measuring baseline injectivity, as 3-8 wt % KCl is a typical clay stabilizer applied in the field. Injectivity testing is typically done with a variety of test fluids. The test fluid may be modeled after fluids that are intended for injection into the subterranean formation in the field.

The test fluid may be formation water/brine taken from various geological formations, which contains different types and concentrations of ions, such as various brines (containing KCl as well as possibly Na, Ca, Mg, Ba, Sr and a number of other ions in various concentrations). The test fluid may be a drilling and/or completion fluid. The test fluid may be a hydraulic fracturing/fracking fluid. The test fluid may be any fluid that is being considered for injection into the subterranean formation or that has been injected into the subterranean formation. The results include the determination of whether a test fluid will cause a change in the permeability of the formation.

The injectivity testing may include a subjective or qualitative injectivity analysis, such as observed swelling, maximum fluid injection pressure, zero fluid flow rate, and general compatibility/incompatibility of the test fluid and the compressed core plug.

The injectivity testing may include a quantitative injectivity analysis, such as the fluid flow rate, change in the fluid injection pressure and production pressure, change in the differential pressure along the compressed core plug, and the confining pressure.

Just as one would use a reservoir core plug for core flood experiments/analysis to study the in-situ displacement performance or relative permeability to oil, water and/or gas, compressed core plug (130) can be used to perform experiments/analysis to study the in-situ displacement performance and/or multi-phase relative permeability, if certain precautions and/or preparations are made, such as restoring the water and oil saturations inside the compressed core plug (130).

In the circumstance where a reservoir core sample plug is available, the reservoir core sample plug can be used to obtain a baseline value for the measured properties (such as porosity, permeability, injectivity, relative permeability, displacement performance). This may confirm/validate the results from the testing of the compressed core plug (130) and/or provide a correction and/or matching model and/or factor.

The described method is more applicable to siliciclastic subterranean formation rocks having a clay content of at least about 5 weight percent, but may be up to and above 15 weight percent. The clay content is understood to help form a more consolidated compressed core plug (130). Further, the clay content tends to magnify compatibility/incompatibility of the test fluid with the compressed core plug (130). One such subterranean formation is the Viking Formation of the Western Canadian Sedimentary Basin.

The described method is more applicable to wells drilled using water based drilling fluids (rather than invert drilling fluids, using hydrocarbon based drilling fluids such as diesel). This is thought to be because it is more difficult to clean the drill cuttings when an invert drilling fluid has been used.

As a relatively abundant supply of drill cuttings are available, a sizeable number of compressed core plugs can be made to carry out multiple coreless injectivity tests for a variety of fluids on a well, where no core sample is available.

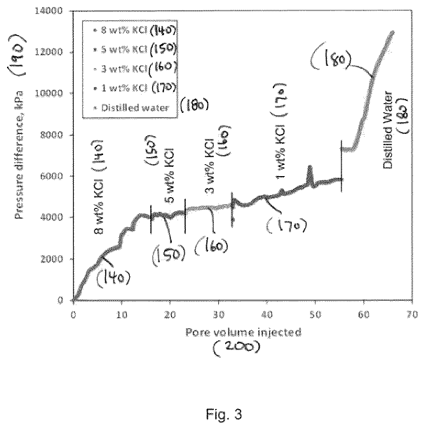

Referring to FIG. 3, coreless injectivity testing of a compressed core plug (130) or plugs made from drill cuttings (100) as described herein provided results similar to those obtained with real reservoir core sample plugs from the same subterranean formation. In addition, permeability and porosity of the compressed core plugs (130) made of drill cuttings (100) as described herein were in the range expected in the formation (in this case the Viking Formation).

FIG. 3 illustrates the pressure difference across the compressed core plug (130) as function of pore volume injected with different KCl concentrations at a constant injection rate of 1 cm³/h for all fluids. Based on Darcy's law of permeability, pressure difference is inversely proportional to permeability provided other parameters, i.e., core diameter and length, fluid viscosity, and fluid injection rate, held constant. Therefore, pressure difference is a good indication of injectivity. The higher the pressure difference, the lower the permeability. When the pressure difference increases for one compressed core plug 130 during an injectivity test while the flowrate is kept constant, it is a clear indication of injectivity loss caused by core damage, i.e., it becomes more difficult to inject the fluid.

Further, FIG. 3 illustrates that the measured pressure difference at 8 wt % KCl injection (140) increased at the beginning when the fluid conditioned the compressed core plug and reached a plateau of about 4000 kPa at a stabilized flow, and remained almost unchanged for 5 wt % KCl injection (150). This indicated the compressed core plug (130) permeability was not compromised with these two fluids. When 3 wt % KCl was injected (160) into the compressed core plug, pressure difference started to increase, and climbed sharply with 1 wt % KCl (170) and distilled water (180). Such results show that the compressed core plug (130) injectivity started to be damaged by mildly low concentration of 3 wt % KCl and deteriorated with reduction of KCl concentration. This result is highly consistent with general understanding on formation damage caused by fresh water on swelling clay rich reservoirs. In FIG. 3, the pressure difference (190) in kPa versus pore volume injected (200) is shown, with the graph indicating 8 wt % KCl (140), 5 wt % KCl (150), 3 wt % KCl (160), 1 wt % KCl (170), and distilled water (180).

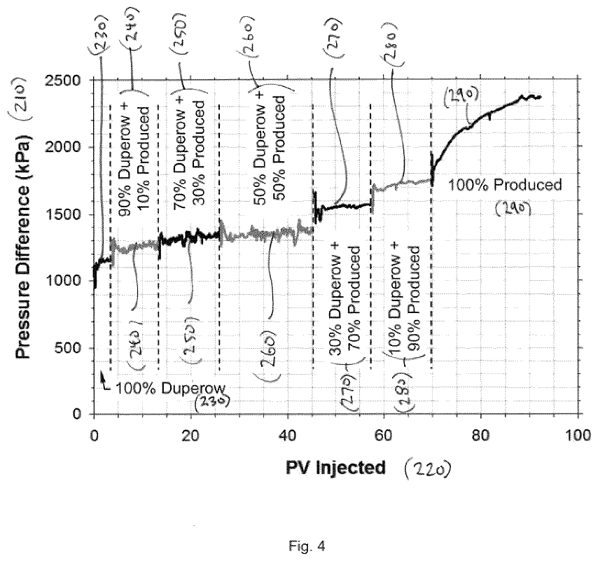

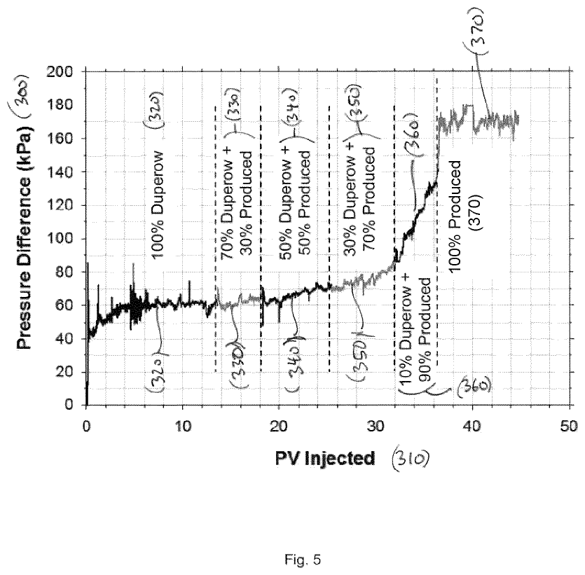

Referring to FIGS. 4 and 5, injectivity testing of a compressed core plug (130) of the present disclosure (FIG. 4) may be compared to injectivity testing of an actual/real reservoir core plug (FIG. 5), obtained from the same well as the drill cuttings used to make the compressed core plug (130). The injectivity testing was conducted using a variety of injection brines. The Duperow brine referenced had a total dissolved solids (TDS) of 57,000 mg/L and the formation water referenced had a total dissolved solids (TDS) of 11,500 mg/L.

Referring to FIG. 4, the pressure difference (210) in kPa versus pore volume injected (220) is shown, with the graph indicating 100% Duperow (230), 90% Duperow+10% Produced (240), 70% Duperow+30% Produced (250), 50% Duperow+50% Produced (260), 30% Duperow+70% Produced (270), 10% Duperow+90% Produced (280), and 100% Produced (290).

Referring to FIG. 5, the pressure difference (300) in kPa versus pore volume injected (310) is shown, with the graph indicating 100% Duperow (320), 70% Duperow+30% Produced (330), 50% Duperow+50% Produced (340), 30% Duperow+70% Produced (350), 10% Duperow+90% Produced (360), and 100% Produced (370).

While the absolute values of pressure difference and pore volume injected differ, plugging appears to occur in both tests when the injected brine contained approximately 70% of produced brine. This information can be used when selecting the composition of the injected brine for the field operations.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A method for assessing a siliciclastic subterranean formation rock petrophysical property, comprising:
providing drill cuttings from the siliciclastic subterranean formation;
cleaning the drill cuttings;
forming a compressed core plug from the drill cuttings;
installing the compressed core plug into a coreholder; and
injecting a test fluid into the compressed core plug in order to assess a compressed core plug petrophysical property,
wherein the compressed core plug petrophysical property represents the corresponding siliciclastic subterranean formation rock petrophysical property, prior to injecting the test fluid into the compressed core plug.

2. The method of claim 1, wherein the siliciclastic subterranean formation rock comprises arenite, wackestone, mudstone, or combinations thereof.

3. The method of claim 1, wherein the petrophysical property is selected from the group consisting of:
fluid injectivity;
in-situ displacement performance;
displacement studies;
displacement efficiency;
relative permeabilities;
multi-phase relative permeabilities;
permeability;
porosity; and
combinations thereof.

4. The method of claim 1, wherein the cleaning comprises washing the drill cuttings with a KCl solution, wherein the drill cuttings were obtained from a drilling operation using a water-based drilling mud.

5. The method of claim 1, wherein the cleaning comprises washing the drill cuttings with a solvent, wherein the drill cuttings were obtained from a drilling operation using an oil-based drilling fluid.

6. The method of claim 5, wherein the solvent comprises benzene, toluene, ethylbenzene, or xylene.

7. The method of claim 6, wherein the solvent is toluene.

8. The method of claim 1, further comprising removing at least a portion of bentonite from the drill cuttings, before cleaning.

9. The method of claim 8, wherein the removing comprises gravity separation.

10. The method of claim 1, further comprising conditioning the drill cuttings prior to forming the compressed core plug.

11. The method of claim 10, wherein the conditioning comprises drying the drill cuttings.

12. The method of claim 11, wherein the conditioning further comprises packing the drill cuttings with a packing fluid.

13. The method of claim 12, wherein the packing fluid comprises a solvent.

14. The method of claim 13, wherein the solvent is an organic or aromatic solvent.

15. The method of claim 14, wherein the organic or aromatic solvent comprises benzene, toluene, ethylbenzene, or xylene.

16. The method of claim 15, wherein the organic or aromatic solvent is toluene.

17. The method of claim 1, wherein forming the compressed core plug comprises placing a quantity of the drill cuttings into a form and applying a pressure to the drill cuttings to compress the drill cuttings to form the compressed core plug.

18. The method of claim 17, wherein the pressure is a selected pressure.

19. The method of claim 18, wherein the selected pressure is in the range of about 3,000 psi to about 10,000 psi.

20. The method of claim 19, wherein the selected pressure is applied for a selected time.

21. The method of claim 20, wherein the selected pressure is substantially 5,000 psi and the selected time is substantially 30 minutes.

22. The method of claim 1, further comprising drying the compressed core plug before injecting the test fluid.

23. The method of claim 2, wherein the petrophysical property comprises injectivity testing, and provides a subjective injectivity analysis, selected from the group consisting of:
observed swelling;
maximum injection pressure;
substantially zero fluid flow rate;
compatibility of the test fluid and the compressed core plug; and
combinations thereof.

24. The method of claim 2, wherein the petrophysical property comprises injectivity testing, and provides a quantitative injectivity analysis, selected from the group consisting of:
fluid flow rate;
change in fluid injection pressure;
change in fluid production pressure;
change in differential pressure along the compressed core plug;
confining pressure; and
combinations thereof.

25. The method of claim 2, further comprising determining a corresponding formation petrophysical property of the siliciclastic subterranean formation from the compressed core plug petrophysical property.

26. The method of claim 1, further comprising reducing a formation core plug obtained from the siliciclastic subterranean formation to obtain the drill cuttings.

27. The method of claim 26, further comprising generating a numerical model, for use with injectivity test results from a compressed core plug, to conform or match the results.

28. The method of claim 1, wherein the siliciclastic subterranean formation rock has a clay content of about 5 wt. percent or more.

29. The method of claim 28, wherein the clay content is about 10 wt. percent or more.

30. The method of claim 29, wherein the clay content is about 15 wt. percent or more.

31. The method of claim 1, wherein installing the compressed core plug into the coreholder comprises stacking a plurality of compressed core plugs into the coreholder.

32. A method for assessing a petrophysical property of a siliciclastic subterranean formation rock having a clay content of about 5 wt. percent or more, comprising:
providing drill cuttings from the siliciclastic subterranean formation;
cleaning the drill cuttings:
a) with KCl if the drill cuttings include water-based drilling fluid; or
b) with a solvent if the drill cuttings include oil-based drilling fluid;
drying the drill cuttings;
packing the drill cuttings with a packing fluid, preferably toluene;
forming a compressed core plug from the drill cuttings by placing a quantity of the drill cuttings into a form and applying a pressure to compress the drill cuttings to form the compressed core plug;
drying the compressed core plug; and
installing the compressed core plug into a coreholder;
injecting a test fluid into the compressed core plug while measuring an injected volume and a differential pressure,
wherein the injectivity of the test fluid into the compressed core plug is representative of the injectivity of the test fluid into the siliciclastic subterranean formation rock.

33. The method of claim 32, The method of claim 1, wherein installing the compressed core plug into the coreholder comprises stacking a plurality of compressed core plugs into the coreholder.

34. A method of forming a compressed core plug for assessing a siliciclastic subterranean formation rock petrophysical property, comprising:
providing drill cuttings from the siliciclastic subterranean formation;
cleaning the drill cuttings:
a) with a KCl solution if the drill cuttings include water-based drilling fluid; or
b) with a solvent if the drill cuttings include oil-based drilling fluid;
drying the drill cuttings;
packing the drill cuttings with a packing fluid, preferably toluene;
forming the compressed core plug from the drill cuttings by placing a quantity of the drill cuttings into a form and applying a pressure to compress the drill cuttings to form the compressed core plug; and
drying the compressed core plug,
wherein the compressed core plug is adapted to undergo core flooding evaluation.

35. The method of claim 34, further comprising installing the compressed core plug into a coreholder.

36. The method of claim 35, wherein installing the compressed core plug into the coreholder comprises stacking a plurality of compressed core plugs into the coreholder.
